(19) United States
(12) Plant Patent Application Publication
(10) Pub. No.: US 2014/0283253 P1
(43) Pub. Date: Sep. 18, 2014

(54) Hydrangea Macrophylla Plant Named 'Little Bo Peep'

(50) ;

(71) Applicant: Joshua H. Kardos, Watkinsville, GA (US)

(72) Inventor: Joshua H. Kardos, Watkinsville, GA (US)

(73) Assignee: Plant Introductions, Inc., Watkinsville, GA (US)

(21) Appl. No.: 13/998,275

(22) Filed: Oct. 17, 2013

Related U.S. Application Data
(60) Provisional application No. 61/784,850, filed on Mar. 14, 2013.

(51) Int. Cl. A01H 5/00

(52) U.S. Cl. PLT/250

(57) ABSTRACT

A new and distinct cultivar of Hydrangea macrophylla plant named ‘Little Bo Peep’, characterized by its remontant (reblooming) trait, flowering on old wood and new growth of the season; small, medium pink mophead inflorescences or medium blue inflorescences in non-aluminum or aluminum based media, respectively; compact, mounded to spreading growth habit; lustrous, dark green foliage; reddish green stems and petioles; and moderate resistance to powdery mildew and leaf spot.

### CROSS REFERENCE TO RELATED APPLICATION

This application claims the benefit of U.S. Provisional Application No. 61/784,850 filed Mar. 14, 2013, the disclosure of which is incorporated by reference in its entirety.

### GENUS AND SPECIES OF PLANT CLAIMED

Hydrangea macrophylla (Thunb.) Ser.

### VARIETY DENOMINATION

‘Little Bo Peep’

### BACKGROUND OF THE INVENTION

The present invention relates to a new and distinct cultivar of Hydrangea macrophylla, a member of the Hydrangeaceae family, hereinafter referred to by its cultivar name ‘Little Bo Peep’. This cultivar is grown primarily as an ornamental for landscape use and for use as a potted plant.

The cultivar originated from a controlled cross-pollination of Hydrangea macrophylla ‘PIIHM-I’ (U.S. Plant Pat. No. 20,176) crossed with Hydrangea macrophylla ‘Mini Penny’ (U.S. Plant Pat. No. 15,744) in Watkinsville, Ga. in 2007, and was selected from the progeny of this cross by continued evaluation for growth habit and improved leaf and flower characteristics. The cultivar ‘Little Bo Peep’ originated and was discovered in a cultivated environment in Watkinsville, Ga.

‘Little Bo Peep’ has been asexually reproduced by softwood cuttings since 2009 in Watkinsville, Ga. The characteristics of the cultivar have been stable and reproduced true to type in successive vegetative generations.

### SUMMARY OF THE INVENTION

‘Little Bo Peep’ has not been observed under all possible environmental conditions. The phenotype may vary somewhat with changes in light, temperature, soil and rainfall without, however, any variance in genotype.

The following traits have been observed and represent the characteristics of the new cultivar. In combination these characteristics distinguish ‘Little Bo Peep’ from all other varieties in commerce known to the inventor. 1) Remontant (reblooming) trait, flowering on old wood and new growth of the season. 2) Small, medium pink mophead inflorescences or medium blue inflorescences in non-aluminum or aluminum based media, respectively. 3) Compact, mounded to spreading growth habit. 4) Lustrous, dark green foliage. 5) Reddish green stems and petioles. 6) Moderate resistance to powdery mildew and leaf spot.

‘Little Bo Peep’ is distinguished from its female parent ‘PIIHM-I’ by its growth habit and inflorescence type. ‘Little Bo Peep’ has a compact, mounded to spreading growth habit and mophead inflorescences, whereas ‘PIIHM-I’ has an overall larger, upright, rounded and spreading growth habit and lacecap inflorescences. ‘Little Bo Peep’ is distinguished from its male parent ‘Mini Penny’ by its growth habit, inflorescence size, flower quantity, and petiole color. ‘Little Bo Peep’ has a compact, mounded to spreading growth habit, smaller inflorescences, more inflorescences per plant, and reddish green stems and petioles, whereas ‘Mini Penny’ has an overall larger, compact, mounded growth habit, larger inflorescences, fewer inflorescences per plant, and green stems and petioles. There are no other cultivars of Hydrangea macrophylla with this combination of traits known to the inventor.

‘Little Bo Peep’ can be compared to the cultivar ‘Pia’ (not patented) but differs in the following characteristics. ‘Little Bo Peep’ reblooms throughout the growing season with medium pink or blue flowers, whereas ‘Pia’ does not rebloom and has darker pink or blue flowers. ‘Little Bo Peep’ has reddish green stems and petioles, whereas ‘Pia’ has green stems and petioles. ‘Little Bo Peep’ has improved cold hardiness, vigor, and resistance to powdery mildew and leaf spot compared to ‘Pia’.

### BRIEF DESCRIPTION OF THE DRAWINGS

The accompanying illustrations show characteristics of the new cultivar in photographs as true to color as is reasonably possible to make in illustrations of this nature. Colors in the photographs may differ slightly from the color values cited in the detailed botanical description which accurately describe the colors of the new Hydrangea.

FIG. 1 illustrates a ‘Little Bo Peep’ plant grown in a container in the absence of aluminum.

FIG. 2 illustrates an inflorescence on ‘Little Bo Peep’ grown in the absence of aluminum.

### DETAILED DESCRIPTION

In the following description, color references are made to The Royal Horticultural Society Colour Chart, 2007 Edition, except where general terms of ordinary dictionary significance are used. Plants used for the description were grown in 11.8 L containers in a medium consisting of composted pine bark that was amended with 45 grams of aluminum sulphate and a pH of 5.7. Plants were grown under 50% shade under outdoor conditions in a nursery in Watkinsville, Ga. Plants were about 2 years old when the description was recorded.
Botanical classification: ‘Little Bo Peep’ is a cultivar of Hydrangea macrophylla. Parentage: The current variety is a progeny from a controlled cross-pollination of Hydrangea macrophylla ‘PIIHM-I’ crossed with Hydrangea macrophylla ‘Mini Penny’. Propagation: Vegetatively by stem cuttings.
Plant description:

The claimed variety is a compact, mounded to spreading deciduous shrub. The original plant, now about 6-years-old in the ground, is about 84 cm in height from the soil level to the top of the inflorescences, and about 198 cm in diameter. Freely branching. The plant is cold hardy in USDA Zones 5 to 9.
Stems: First year stems have a diameter of about 4 mm, a round shape, and a stout, glabrous and lustrous texture with very fine pubescence. Exfoliation: on second year and older stems, flaky and stringy. Internodes have a length of about 3.5 cm. The stem color is 178A maturing to 144C. Second year stems have a diameter of about 6 mm or more and are N199D in color.
Vegetative buds: Arrangement: opposite; Shape: ovoid; Size: about 2 mm in length, about 1 mm in width; Color: 144B.
Leaves:
Size: About 16.6 cm in length and about 7.5 cm in width. Shape: ovate, with acuminate apex, cuneate base and serrate margin. Texture: thick and leathery. Color: emerging leaves are 146A on the upper and lower surfaces; mature leaves on the upper surface are 147A, and 147B on the lower surface.
Venation: Pinnate, veins are 144C on upper and lower surfaces.
Petiole: About 2.4 cm in length and about 3.5 mm in diameter, glabrous texture, and 178A in color.
Flowers:
Inflorescence bloom period: Early summer to first frost in fall. An inflorescence contains about 62 individual fertile flowers. An inflorescence contains about 280 individual sterile florets. Inflorescence shape and size: mophead, about 7.2 cm in height and about 8.9 cm in diameter. The inflorescence is effective for about 8 weeks. The peduncle is about 4.6 cm in length, is finely pubescent, and is 144C in color. The pedicels are about 1 cm in length, finely pubescent, and 144C in color.
Flower bud size: About 2 mm in length, about 2 mm in diameter. Flower bud shape: round; Color: N88D. Sterile florets are about 2 cm in diameter. Each floret contains 4 sepals about 9 mm in length and about 8 mm in width. Sterile floret shape: ovoid with obtuse apex, acute base, and entire margin; Texture: smooth with no pubescence; Color at maturity: the upper surface is N88C and the lower surface is 91B.
Petals (fertile flowers): Size: 5 petals per flower, about 2.5 mm in length and about 1.5 mm in width; Shape: ovate, with acuminate apex, truncate base, and entire margin; Texture: smooth with no pubescence; Color: at peak of bloom the upper surface of the petals is 92B and the lower surface is 91B.
Number of stamens: 6 to 10. Anthers: about 1 mm in length and about 0.5 mm in width, 91B in color. Filaments: about 2.5 mm in length and 91C in color. Pollen: NN155C in color, produced in moderate quantities. Pistil: superior, about 2.5 mm in length and about 1.5 mm in width, 91B in color. Stigma: 2 or 3 per pistil, round in shape and 91C in color. Style: about 1.5 mm in length and tubular in shape, 91B in color.
Fruit: The capsule fruit is ovoid, about 3 mm in length and about 2 mm in width. Color at maturity is 200C. The number of fruit per infructescence varies widely. Seed: The seeds are round, about 0.5 mm in length and about 0.5 mm in width, close to 199B, and each capsule contains about 50 seeds.
Disease/pest resistance: Moderately resistant to powdery mildew and leaf spot. No other pest or disease resistance/susceptibility has been observed. ‘Little Bo Peep’ is cold hardy from USDA Cold Hardiness Zones 5-9.

I claim:

1. A new and distinct Hydrangea macrophylla plant named ‘Little Bo Peep’, substantially as illustrated and described herein.
